(19) United States
(12) Patent Application Publication
(10) Pub. No.: US 2025/0322964 A1
(43) Pub. Date: Oct. 16, 2025

(54) AI-ASSISTED, DATA DRIVEN, REAL TIME POSTURE DETECTION FOR PHYSIOTHERAPY, FALL PREVENTION, AND FRAILTY ASSESSMENTS

(71) Applicant: Arizona Board of Regents on Behalf of the University of Arizona, Tucson, AZ (US)

(72) Inventors: Kavan Hazeli, Tucson, AZ (US); Rylie Watson, Tucson, AZ (US); Xuejing Wang, Tucson, AZ (US)

(21) Appl. No.: 19/175,990

(22) Filed: Apr. 10, 2025

Related U.S. Application Data
(60) Provisional application No. 63/632,447, filed on Apr. 10, 2024.

(51) Int. Cl. G16H 50/50; A61B 5/00; A61B 5/11; G06T 3/40; G06T 7/00; G06T 7/73; G06T 11/00; G06T 13/40; G16H 10/60; G16H 50/20

(52) U.S. Cl. CPC G16H 50/50; A61B 5/112; A61B 5/4561; A61B 5/7275; A61B 5/7425; G06T 3/40; G06T 7/0016; G06T 7/74; G06T 11/00; G06T 13/40; G16H 10/60; G16H 50/20; G06T 2200/24; G06T 2207/10016; G06T 2207/30004; G06T 2207/30196; G06T 2210/41

(57) ABSTRACT

An AI-assisted, data driven, real time posture detection system for physiotherapy, fall prevention, and/or frailty assessments. The system uses a pre-trained pose detection model and video images from multiple angles captured by multiple cameras to determine the three-dimensional locations of user joints in real time. In embodiments, the system calculates qualitative metrics (e.g., range of motion, ankle dorsiflexion, Q-angle, hip-knee-ankle alignment, gait speed, etc.) to determine whether the user has suffered or is at risk of an injury (e.g., ACL tear, patellar tendonitis, hip fracture, etc.) and/or whether a physiological condition has worsened or improved over time (e.g., after surgery or physical therapy). In some embodiments, the system constructs a digital twin of the user and displays a visual representation of the digital twin performing idealized movements (e.g., proper form for exercise or physical therapy) to provide real-time instruction and feedback to improve the physiological condition of the user.

### CROSS-REFERENCE TO RELATED APPLICATIONS

This application claims priority to U.S. Prov. Pat. Appl. No. 63/632,447, filed Apr. 10, 2024, which is hereby incorporated by reference in its entirety.

### FEDERAL FUNDING

None

### BACKGROUND

Physiotherapy is a critical component of health care, as it addresses a vast array of needs in a variety of settings and life stages, including assisting the elderly in maintaining mobility and enhancing the quality of life, facilitating the recovery process following surgery or injury, and optimizing athletic performance to achieve peak physical condition.

As a result of sports, fitness, and other motion injuries, physical therapy is required in order to maintain mobility, decrease pain, and manage injury. The most common injuries, admitted into hospitals, are dislocations, sprains, and strains (45.4%), musculoskeletal or tendon injury (16%), fractures (14.4%), and open wounds (17.1%). The most common areas of injury are the knee (39%), ankle (28%), shoulder (22%), and back (18%). The most common exercises that cause injury are free weights/machine usage (42.4%). Though many fitness injuries remain unreported as a result of individuals not seeking medical attention, but rather, varying their training intensity or duration. The lack of reporting acute injuries leads to the underestimation of fitness injuries that occur each year.

For the elderly, home-friendly health monitoring devices and care solutions are expected to grow in popularity in the market. An illustration of this is the development of devices and methods for monitoring and caring for frailty, in the home environment. As a very common difficulty faced by the elderly population, frailty is always associated by mortality, dependence, and unfavorable health outcomes. Physicians and researchers conduct studies to detect frailty and intervene in a timely manner in order to mitigate the negative effects of frailty. Frailty is diagnosed through the application of laboratory-based motion-tracking systems, impairment in daily physical activities (PA), and additional semi-objective tests. As of now, objective instrumented assessments used for in-home frailty screening have not undergone sufficient development and validation.

As of 2023, the physical therapy market accounted for $53.08 billion dollars. While the length of treatments varies based on each individual's unique needs it is generally accepted that minor injuries require 1-3 sessions of physiotherapy, where soft tissue injuries require 6-8 weeks and chronic conditions over 2 months. The demand for physiotherapy services is anticipated to increase consistently in the foreseeable future, as public awareness regarding the significance of physiotherapy in maintaining physical health gradually grows.

At present, there is an absence of health devices capable of methodically quantifying the efficacy of physiotherapy. Some software application use pose detection to offer basic exercise guidance. However, those systems only perform pose detection in two dimensions, severely limiting their functionality. Additionally, those software applications do not provide injury or injury risk assessment, therapeutic evaluation over time, frailty assessment, or gait assessment for fall prevention.

Meanwhile, getting to real-time quantification is an even greater challenge. Frequently, physiotherapists devote a significant amount of time to analyzing and reviewing videos of patients. Nevertheless, these videos mostly focus on particular instances and patterns of behavior, disregarding the daily routine movements and activities of the patients potentially wasting considerable time for therapists with minimal to no benefit.

### SUMMARY

The disclosed system combines computer vision algorithms, artificial intelligence models, and statistical tools to convert high-dimensional healthcare monitoring videos into low-dimensional data that can be used for real time, data driven physiotherapy, fall detection, and frailty assessment.

U.S. patent application Ser. No. 18/429,089, which is hereby incorporated by reference, describes using video monitoring and pose detection to monitor and assess the real-time stability of a user, for example by determining whether the center of mass of the user is located over the base of support of the user. The disclosed system uses similar pose detection methods to identify the angular position, separation, and height of major joints of the user. The disclosed system calculates qualitative metrics of interest to the specific application, for instance the angle of a virtual line between two joints of the user (e.g., a foot index and a shoulder) relative to the ground (or the gravitational field perpendicular to the ground), the angle of a virtual line between two joints of the user (e.g., an elbow and a wrist) relative to a virtual line between two other joints of the user (e.g., the left and right hip), the angle created by two virtual lines from two joints of the user (e.g., a shoulder and a wrist) to a third joint of the user (e.g., the elbow between the shoulder and the wrist). The disclosed system may also include statistical tools for determining whether potential data of interest is correlated with certain outcomes (e.g., falls, physiotherapy improvement, etc.).

By capturing and quantifying data during physiotherapy, the disclosed system enables practitioners to make data-driven assessments of the physiotherapy of users, which may be more accurate and more cost effective than watching users (or videos of users) performing prescribed therapies. Additionally, by capturing and quantifying data of users over time, the disclosed system can be used to make data-driven assessments of the efficacy of prescribed interventions (e.g., surgery, physical therapy, etc.). Additionally, because the disclosed system may capture video images of users at home (like fall prevention system of U.S. patent application Ser. No. 18/429,089), the disclosed system also enables practitioners to assess of the routine movements and activities of users outside the clinical setting.

The disclosed system can also provide feedback to users and/or practitioners in real time. For example, the disclosed system provides functionality to specify a digital twin having similar features as the user (e.g., weight, body shape, angles of freedom for specific circumstances such as a ligament or tendon injury, etc.) and functionality for practitioners to specify optimized therapies. The disclosed system can then graphically depict the digital twin of the user performing the therapy specified by the practitioner, enabling the user to better recognize and perform the specified therapy by mimicking the movements of the digital twin. Additionally, the disclosed system enables practitioners to make data-driven determinations as to whether exercises are being performed correctly and safely. Additionally, the disclosed system can graphically depict both the user and the digital twin, providing the user with real-time feedback on whether the user is correctly and safely performing the prescribed therapy. Accordingly, the disclosed system enables users to receive physical therapy that has been tailored to their specific body and/or injuries even outside a clinical setting (e.g., at home).

When employed in an at-home environment, the disclosed system can also be used to assess the frailty of users based on data (e.g., gait data) captured as the user performs everyday activities. For instance, the disclosed system can be used to model two of the five measured items for phenotypic frailty: slowness from usual gait speed and low activity. By capturing gait data, for instance, the disclosed system can determine whether a user is moving slower than usual as they walk into and out of the field. Additionally, the disclosed system can characterize each activity of the user (e.g., standing, walking, lying down) and determine whether a user has low activity by comparing the amount of time the user spends doing each activity. Additionally, the described system can be used to assess the user performing a short physical performance battery test (e.g., a chair rise test, a balance test, etc.) and score the performance of the user based on time it takes the user to perform the complete the short physical performance battery test.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a diagram of an architecture of the disclosed system according to exemplary embodiments.

FIG. 2 is a block diagram of the disclosed system according to exemplary embodiments.

FIG. 3A illustrates the Silverskiold test is used to assess for gastrocnemius equinus.

FIG. 3B illustrates a quantitative, real-time assessment of Dynamic Gastrocnemius Equinus Contracture (dGEC) using the disclosed system according to an exemplary embodiment.

FIG. 4A illustrates a quantitative, real-time assessment of the risk of anterior cruciate ligament (ACL) tear using the disclosed system according to an exemplary embodiment.

FIG. 4B illustrates a quantitative, real-time, early detection of patellar tendonitis using the disclosed system according to an exemplary embodiment.

FIG. 5A illustrates real-time therapeutic instruction provided the system according to an exemplary embodiment.

FIG. 5B illustrates example qualitative metrics according to an exemplary embodiment.

FIG. 5C illustrates therapeutic evaluation provided by the system according to an exemplary embodiment.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

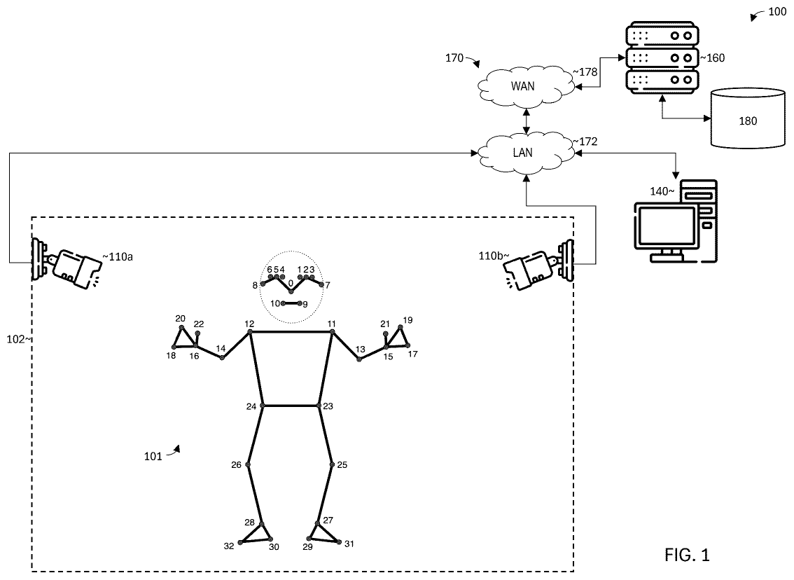

FIG. 1 is a diagram of an architecture 100 of the disclosed system according to exemplary embodiments.

As shown in FIG. 1, the architecture 100 of the disclosed system includes multiple video cameras 110a, 110b, etc. (generically and collectively referred to herein as video camera(s) 110) in communication with a local computer 140 and/or remote server 160, for example via one or more communication networks 170 (e.g., a local area network 172 and/or a wide area network 178 such as the internet). Each of the local computer 140 and/or the remote server 160 include a hardware computer processor that executes instructions stored on non-transitory computer readable storage media to perform the functions described herein. The local computer 140 and/or remote server 160 includes or is in communication with non-transitory computer readable storage media 190, which may be realized as remote storage (i.e., cloud storage) or local to the environment 102 of the user 101.

The video cameras 110 capture image data of an environment 102 of a user 101. The images of each user 101 includes a number of facial landmarks indicative of the locations of facial features—for example, landmarks indicative of the nose 0, left eye (inner) 1, left eye 2, left eye (outer) 3, right eye (inner) 4, right eye 5, right eye (outer) 6, left ear 7, right ear 8, mouth (left) 9, and mouth (right) 10 of the user 101—and a number of landmarks indicative of the locations of joints—for example, landmarks indicative of the left shoulder 11, right shoulder 12, left elbow 13, right elbow 14, left wrist 15, right wrist 16, left pinky finger 17, right pinky finger 18, right index 20, left thumb 21, right thumb 22, left hip 23, right hip 24, left knee 25, right knee 26, left ankle 27, right ankle 28, left heel 29, right heel 30, left foot index 31, and right foot index 32.

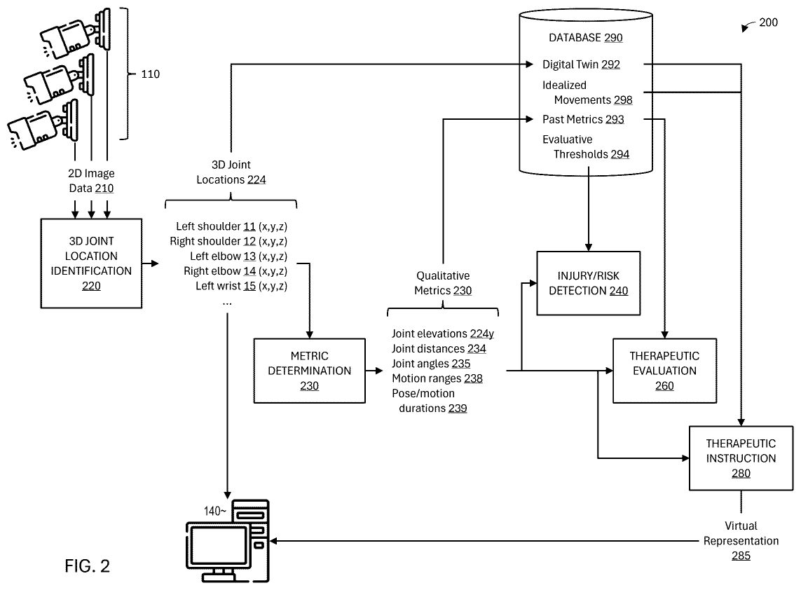

FIG. 2 is a block diagram of the disclosed system 200 according to exemplary embodiments.

In the embodiment of FIG. 2, the system 200 includes a database 290 (stored, for example, on the computer readable storage media 190) and a three-dimensional joint identification module 220, a metric determination module 230, an injury/risk detection module 240, a therapeutic evaluation module 260, and a therapeutic instruction module 280 (realized as software instructions executed by the local computer 140 and/or the remote server 160).

The three-dimensional joint identification module 220 that uses two-dimensional image data 210 output by multiple video cameras 110 (e.g., two or three video camera 110) to identify the relative locations 224 of joints of the user 101 in three dimensions. For instance, the three-dimensional joint identification module 220 may use a pre-trained pose detection model (e.g., MediaPipe Pose, a powerful machine learning solution developed by Google that excels at tracking human body poses in real-time) to infer landmarks in the image data 210 output by each video camera 110 indicative of joints of the user 101. Using those landmarks as well as the relative position of each of the cameras 110, the three-dimensional joint identification module 220 estimates the relative locations 224 of those joints in three dimensions.

The metric determination module 230 uses the three-dimensional joint locations 22 of the user 101 to calculate qualitative metrics 230 used by the system 200 as described below. The metrics 230 may include static metrics 230, such as the elevation 224y of each joint (e.g., the location 224 of each joint along the y-axis perpendicular to the ground), distances 234 between individual joints, joint angles 235, etc. The joint angles 235 may include angles 235 formed by a virtual line intersecting two joints of the user 101 (e.g., the right elbow 14 and the right wrist 16) relative to the ground (or the gravitational field perpendicular to the ground), angles 235 formed by a virtual line intersecting two joints of the user 101 (e.g., the right hip 24 and the right knee 26) relative to a second virtual line intersecting two other joints of the user 101 (e.g., the right knee 26 and the right ankle 28), and/or angles 235 formed by two virtual lines from two joints of the user (e.g., the left shoulder 11 and the left wrist 15) to a third joint of the user (e.g., the left elbow 13). The metrics may also include dynamic metrics, for example ranges 238 of motion, durations 239 of poses and/or motions, etc.—determined using the three-dimensional joint locations 224 of the user 101 over time.

As described in more detail below, the three-dimensional joint locations 224 of the user 101 are used to construct a digital twin 292 of the user 101. The digital twin 292 of each user 101 includes information indicative of the size of the user 101 (e.g., the distances 234 between joints). In some embodiments, digital twins 292 may also include other health information (e.g., from electric health records) identifying movement constraints of the user 101, previous interventions (e.g., surgery, physical therapy, etc.), and/or other health conditions of the user 101.

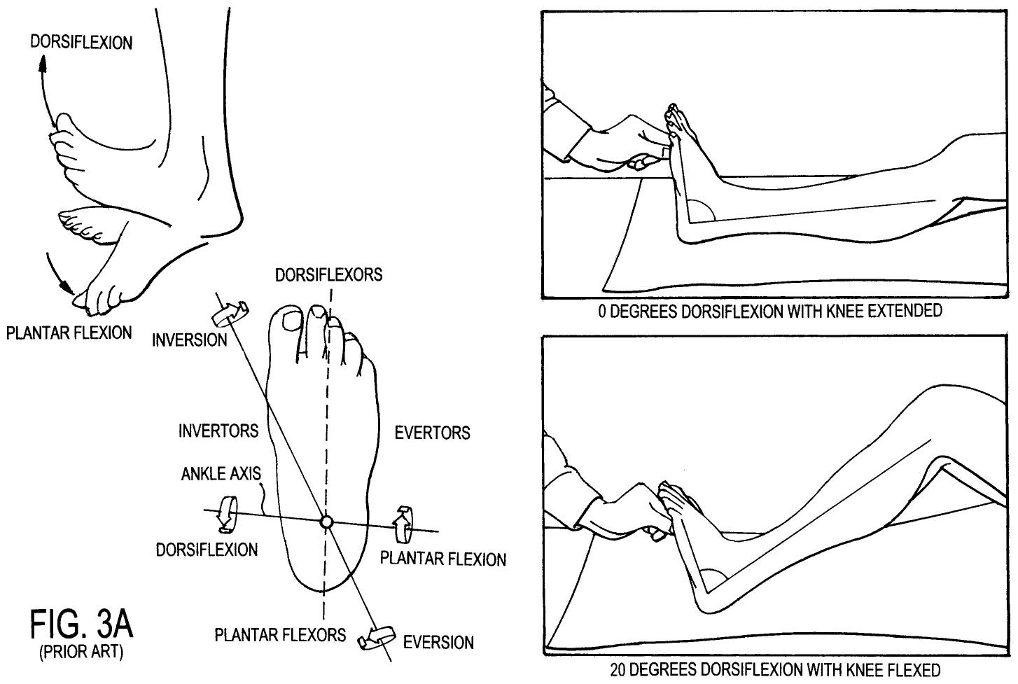

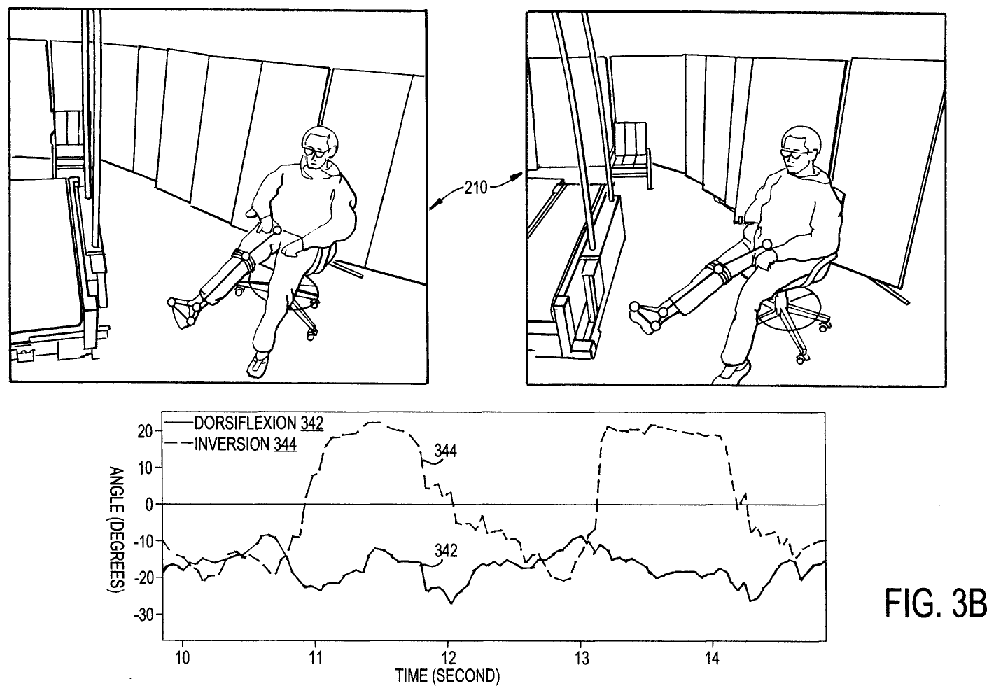

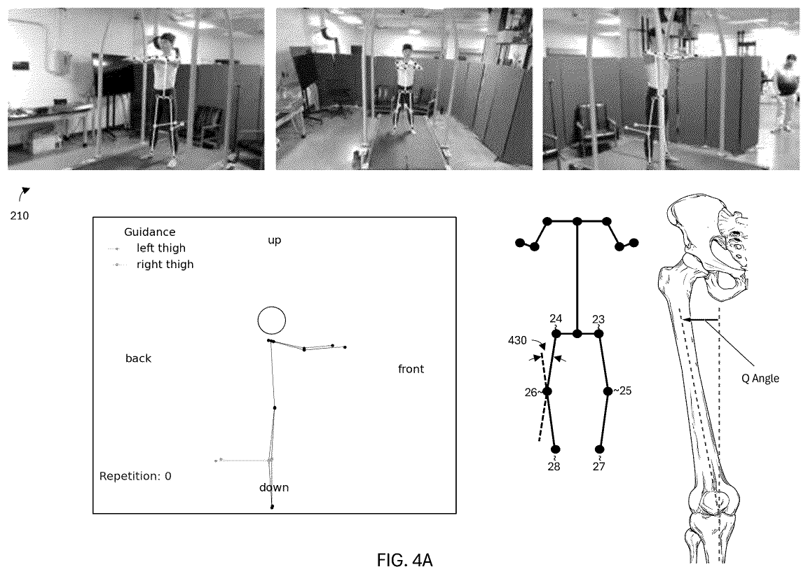

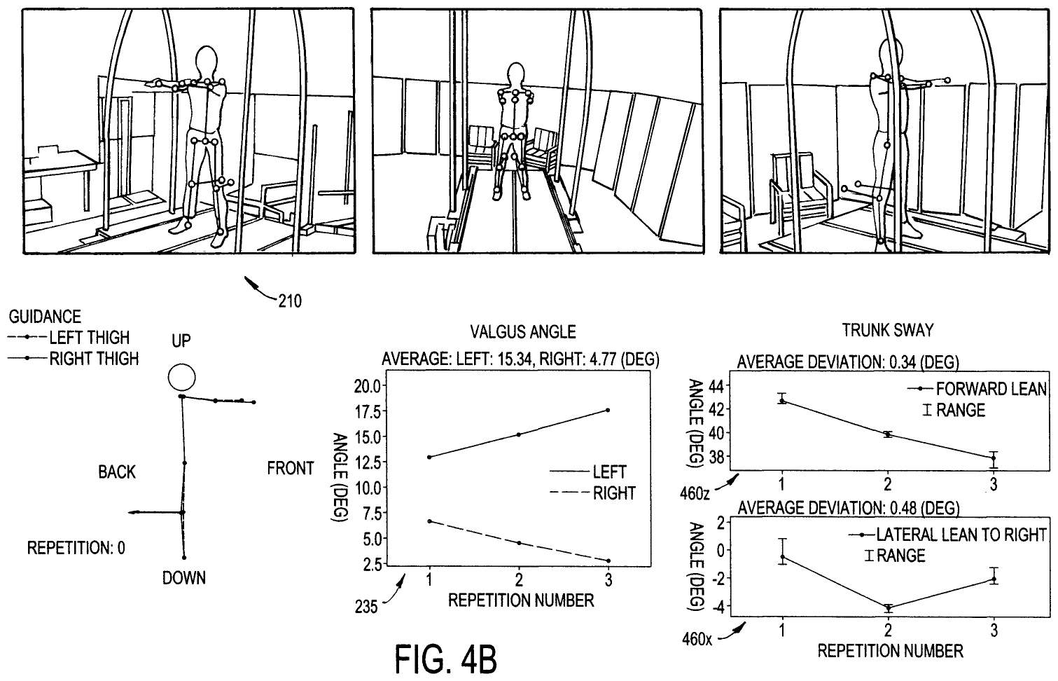

As described in more detail below with reference to FIGS. 3A-4B, the injury/risk detection 240 quantitatively assesses potential injuries of the user 101 (and/or the risk of injuries of the user 101) by comparing the qualitative metrics 230 to evaluative thresholds 294 identified by physiotherapists or other medical experts.

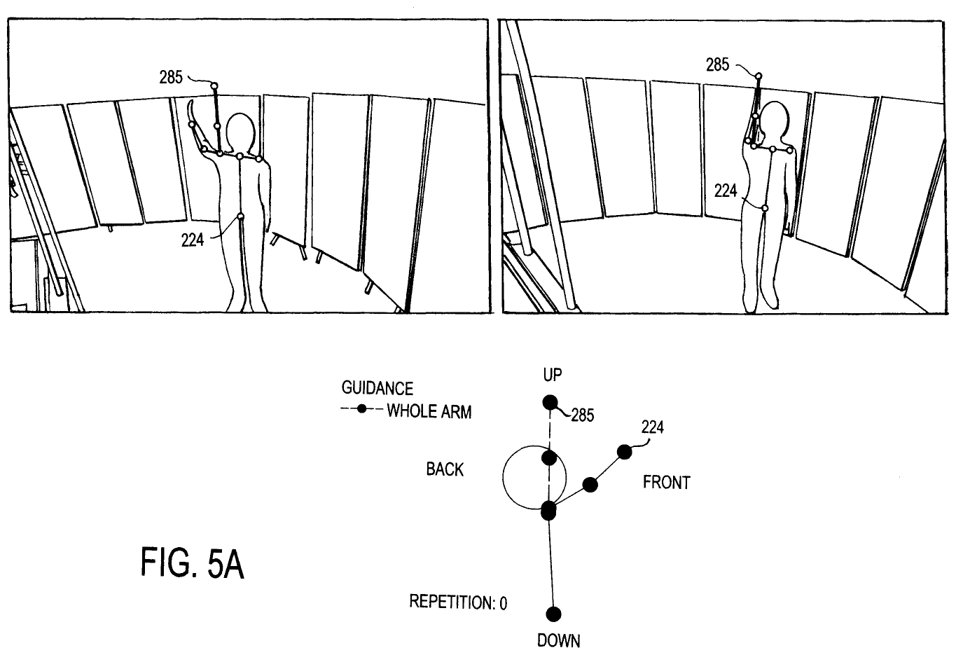

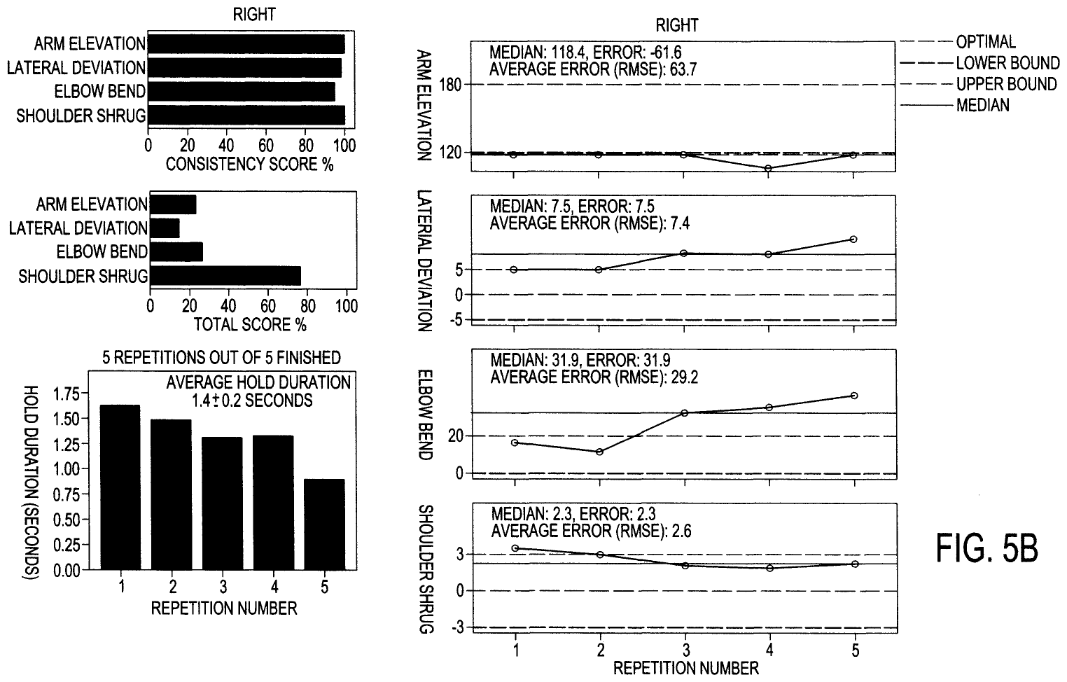

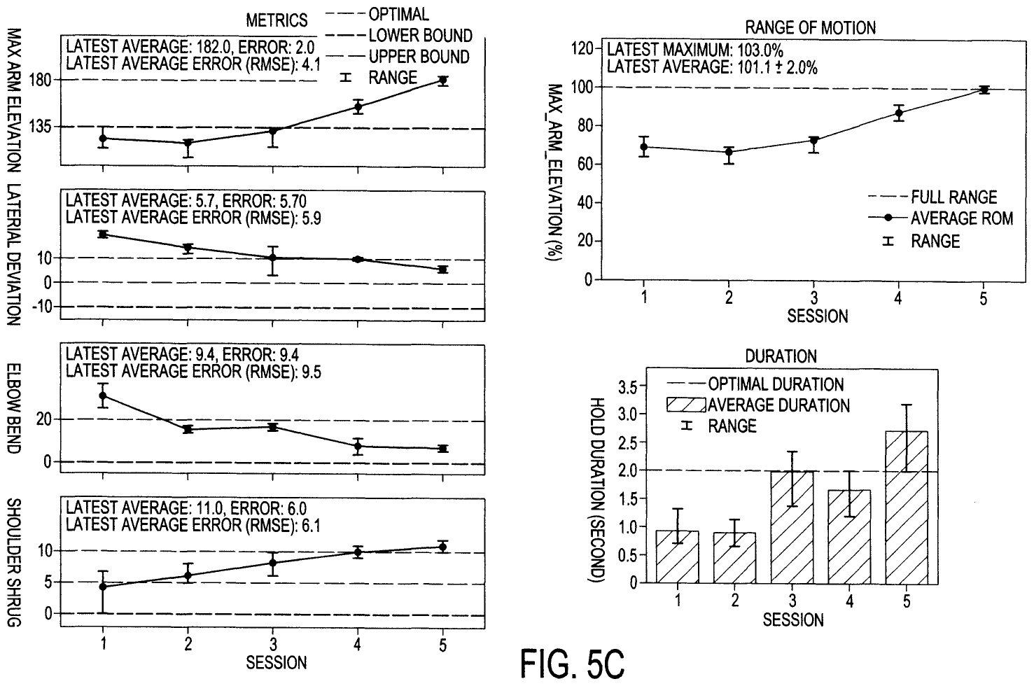

As described in more detail below with reference to FIGS. 5A-5B, the therapeutic instruction module 280 provides real-time instruction to the user 101 by outputting a virtual representation 285 of the digital twin 292 of the user 101 performing idealized movements 298 defined by physiotherapists or other medical experts.

As described in more detail below with reference to FIGS. 5C, the therapeutic evaluation module 260 can be used to assess the user 101 by comparing the qualitative metrics 230 to past metrics 293 of the user 101, for instance the same qualitative metrics 230 captured prior to an intervention (e.g., surgery, physical therapy, etc.).

### Injury/Risk Detection

FIGS. 3A and 3B illustrate a quantitative, real-time assessment of Dynamic Gastrocnemius Equinus Contracture (dGEC) using the disclosed system 200 according to an exemplary embodiment.

As shown in FIG. 3A and described in Latt et al. (2020),¹, the Silverskiold test is used to assess for gastrocnemius equinus. The maximum passive ankle dorsiflexion is compared with the knee extended to the knee flexed. The difference between dorsiflexion in these two positions is the contribution of the gastrocnemius to the equinus contracture because the gastrocnemius crosses both the ankle and the knee joints whereas the other plantarflexors of the ankle do not. Dorsiflexion of less than 10 degrees with the knee extended or a difference of greater than 10 degrees confirms the presence of gastrocnemius equinus contracture. ¹Latt et al., Evaluation and Treatment of Chronic Plantar Fasciitis, Foot Ankle Orthop. 2020 Feb. 13; 5 (1): 2473011419896763. doi: 10.1177/2473011419896763

As shown in FIG. 3B, the disclosed system 200 can be used to quantify the ankle dorsiflexion 342 of the 101 in real time. By quantifying dorsiflexion 342 along the ankle axis, the system 200 can be used to diagnose and/or assess, for example, equinus deformity, Achilles tendinopathy/contracture, gastrocnemius/soleus contracture, ankle osteoarthritis, anterior ankle impingement, posterior tibial tendon dysfunction (PTTD), neuromuscular conditions, etc.

Critically, because the system 200 uses image data 210 from multiple video cameras 110 to identify joint locations 224 in three dimensions, the disclosed system 200 can quantify both dorsiflexion 342 along the ankle axis as well as inversion 344 along the subtalar axis. Accordingly, by quantifying both dorsiflexion 342 along the ankle axis and inversion 344 along the subtalar axis, the system 200 can be used to diagnose and/or assess, for example, chronic or acute ankle instability, complex regional pain syndrome, post-traumatic arthritis, certain neuromuscular conditions (e.g., cerebral palsy or stroke), etc. Finally, by quantifying inversion 344, the system 200 can be used to diagnose and/or assess, for example, injuries to the anterior talofibular ligament (ATFL), the calcaneofibular ligament (CFL), or the posterior talofibular ligament (PTFL), subtalar instability, calcaneal fractures, and/or peroneal tendon injuries.

FIG. 4A illustrates a quantitative, real-time assessment of the risk of anterior cruciate ligament (ACL) tear using the disclosed system 200 according to an exemplary embodiment.

The quadriceps angle (or “Q-angle”) is a measurement of the angle formed by the quadriceps muscles and the patellar tendon. Normal Q-angles range from 10-14 degrees in males and 15-17 degrees in females. Individuals with a higher Q-angles are prone to dynamic knee valgus (inward knee movement during activities such as jumping, landing, or cutting), which is a significant risk factor for ACL injuries. The Q-angle is formed by two lines: one line drawn from the anterior superior iliac spine (ASIS) to the midpoint of the patella and another line drawn from the midpoint of the patella to the tibial tubercle.

As shown in FIG. 4A, using image data 210 of the user 101 (e.g., while performing squats), the system 200 can estimate the Q-angle 430 of the user 101 by using the location of the hip 23 or 24 as a proxy for the location of the ASIS, the knee 25 or 26 as a proxy for the center of the patella, and the line drawn from the knee 25 or 26 to from the ankle 27 or 28 as a proxy form the line drawn from the midpoint of the patella to the tibial tubercle.

FIG. 4B illustrates a quantitative, real-time, early detection of patellar tendonitis using the disclosed system 200 according to an exemplary embodiment.

Patellar tendonitis can be caused by poor hip-knee-ankle alignment, limited ankle dorsiflexion 342 (which may be quantified by the disclosed system 200 as described above with reference to FIG. 3B), and trunk sway. For hip-knee-ankle alignment, a normal range is typically 14 degrees, while a joint angle 235 of greater than 20 degrees is indicative of a valgus knee. As shown in FIG. 4B, similar image data 210 as illustrated in FIG. 4A can be used to quantify the joint angle 235 formed by the hip 23 or 24, knee 25 or 26, and ankle 27 or 28 as well as forward displacement 460z and lateral displacement 460x indicative of trunk sway.

Accordingly, by comparing the qualitative metrics 230 of the user 101 to one or more evaluative thresholds 294 (e.g., a threshold ankle dorsiflexion 342, a threshold Q-angle 430, a threshold hip-knee-ankle joint angle 225, etc.), the disclosed system 200 can be used detect or assess injuries (e.g., gastrocnemius equinus, patellar tendonitis, etc.) and/or the risk of injuries (e.g., ACL tears). The evaluative thresholds 294 may include for example, thresholds 294 indicating that the user 101 would benefit from an intervention (e.g., surgery, physical therapy). Additionally, the evaluative thresholds 294 may also include additional thresholds 294 indicating that one or more interventions would not improve the condition of the user 101. The evaluative thresholds 294 may be provided by physiotherapists or other medical experts, identified in published literature, etc.

While the system 200 is described herein as calculating example qualitative metrics 230 to assess whether the user 101 has suffered or is at risk of example injuries, those of ordinary skill in the art will recognize that the disclosed system 200 can be used to determine whether the user 101 has suffered or is at risk of any injury and, to do so, the system 200 may calculate additional qualitative metrics 230 not specified herein.

### Therapeutic Instruction

FIG. 5A illustrates real-time therapeutic instruction provided the system 200 according to an exemplary embodiment.

As shown in FIG. 5A, the system 200 can be used to provide a virtual representation 285 of the digital twin 292 of the user 101 performing idealized movements 298 defined by physiotherapists or other medical experts. As shown in FIG. 5A, for instance, the system 200 may overlay both the joint locations 224 and the idealized movements 285 over the image data 210 of the user 101. Accordingly, in those embodiments, the system 200 provides the user 101 with real-time instruction for performing physical therapy and/or exercises to prevent and/or recover from injury and improve physical health.

The system 200 may store generic idealized movements 298 for users 101 of all sizes, which are then scaled by the system 200 to visually represent those idealized movements 298 as performed by an individual having the dimensions stored as part of the digital twin 292 of the user 101. Additionally, in embodiments where the digital twin 292 of the user includes other health information of the user 101 (e.g., motion limitations), the system 200 may modify the generic idealized movements 298 to account for those conditions.

FIG. 5B illustrates example qualitative metrics 230 (arm elevation, lateral deviation, elbow bend, shoulder shrug, and hold duration) that may be calculated based as the user 101 follows the therapeutic instruction output by the visual representation 285 illustrated in FIG. 5A.

While the system 200 is described herein as providing example idealized movements 298, those of ordinary skill in the art will recognize that the disclosed system 200 can be used to generate any virtual representation 285 of the digital twin 292 of the user 101 performing any idealized movements 298.

### Therapeutic Evaluation

FIG. 5C illustrates example qualitative metrics 230 (arm elevation, lateral deviation, elbow bend, shoulder shrug, range of motion, and hold duration) calculated based on the three-dimensional joint locations 224 of the user 101 over time. As briefly mentioned above, the qualitative metrics 230 of the user 101 can be compared to the past qualitative metrics 293 of the user 101 to evaluate the effectiveness of a therapeutic treatment. For instance, to evaluate the effectiveness of an intervention (e.g., surgery, physical therapy), qualitative metrics 230 captured after an intervention can be compared to the past qualitative metrics 293 of the user 101 captured prior to the intervention. Notably, improvement in the mobility of the user 101 may not be uniform across multiple axes. Accordingly, by identifying joint locations 224 of the user 101 in three dimensions, the disclosed system 200 is able to calculate quantitative metrics 230 that quantify and differentiate between improvements along multiple axes.

While the system 200 is described herein as comparing example qualitative metrics 230 over time, those of ordinary skill in the art will recognize that the disclosed system 200 can be used to compare additional qualitative metrics 230 not specified herein.

### Fraud Detection

Additionally, by comparing qualitative metrics 230 of the user 101 to past qualitative metrics 293 of the user 101, the disclosed system 200 may be used to detect fraud by the user 101. For instance, a user 101 may contend that they are incapable of performing certain motions. However, individuals are not capable of exhibiting limitations in their range of motion consistently over time. Accordingly, by comparing the comparing qualitative metrics 230 of the user 101 to past qualitative metrics 293 of the user 101, the disclosed system 200 can identify deviations in those metrics 230 that are inconsistent with an individual having the physical limitation claimed by the user 101.

### Frailty Assessment

Frailty is defined as a clinical syndrome driven by age related biological changes which drive physical characteristics of frailty and adverse outcomes, mostly conceptualized as a pre-disability state. Frailty was first described by Fried in which its physical characteristics or phenotype was identified as the presence of three or more of five components: weakness (grip strength), slowness (slow gait), shrinking (weight loss), exhaustion (subjective), and low activity. Upon assessment from a physician, they may then expand the Fried method to use the Clinical Frailty Scale (CFS), which provides a summary for assessing frailty and fitness of patients.

Frailty assessments are crucial for the elderly for early identification of vulnerability, prevention of functional decline, reducing hospitalization, and many other patient-centered care plans and activities. Frailty assessments typically involve a variety of activities and tools, combining clinical evaluations, physical measurements, and self-reported information. However, due to the high demands on settings, personnel, and equipment, these requirements result in a lack of frailty assessments specifically tailored to daily behaviors.

The disclosed system 200 can address the absence of daily frailty assessments by employing machine learning models that analyze the relative positions and angles of major joints during daily activities. For instance, the disclosed system 200 can be used to model two of the five measured items for phenotypic frailty, slowness from usual gait speed and low activity (Cardiovascular Health Study). With the disclosed system 200 being employed in an at-home environment 102, the system 200 can collect vast amounts of gait data as the user completes everyday activities. From that data, the disclosed system can determine whether a user 101 is moving slower than usual as they walk into and out of the field of view of the video cameras 110.

Additionally, algorithms can be used to classify user activities using the image data 210 of the user 101. In those embodiments, low activity may be extrapolated from those activity determinations (grouping activities into categories that include standing, walking or lying down) by measuring and comparing the amount of time the user 101 spends doing each. Additionally, that data can be paired with metabolic estimations to calculate the caloric expenditure of user 101, which can be used to then alert physicians of changes in activity (Men: <383 kcal/week, Women: <270 kcal/week). Furthermore, the described system 200 can be used as a means to assess short physical performance battery tests (chair rise tests and balance tests), which can then be scored on a scale of 1-4 based on the time it takes to complete. Subsequently, data collected over the entire time the system 200 is employed is beneficial in further understanding how frailty develops as a result of age, which can help physicians better understand and quantify frailty characteristics outside of the questionnaires and in person visits that are commonly used today.

### Gait Monitoring and Fall Prevention

Falls and hip fractures are a significant public health problem, particularly for older adults and individuals with certain medical conditions (e.g., Parkinson's disease, stroke, arthritis, and neurological disorders). Hip fractures often require surgery and extensive rehabilitation and can lead to long-term disability, loss of independence, and decreased quality of life. Meanwhile, his fractures are associated with an increased risk of death, especially in older adults.

Subtle gait and mobility changes often occur months before a fall. Early signs include reduced stride length, slower walking speed, and uneven weight distribution. By calculating qualitative metrics 230 and comparing those metrics to the past metrics 293 of the user, the disclosed system 200 can periodically or even continuously assess the gait of the user 101 (e.g., if deployed in an in-home environment 102) and identify early signs of a fall. Accordingly, the system 200 can be used to identify when to implement preventive interventions (e.g., targeted physical therapy, strength and balance training, or in-home environmental modifications) to prevent falls and hip fractures.

### Example Benefits

By quantifying range of motion 238 and other qualitative metrics 230, the disclosed system 200 can be used in the assessment of countless physiological conditions and the diagnosing of countless physiological impairments related to joint functionality.

When used for providing joint functionality and other orthopedic assessments, for example, the disclosed system 200 assist in restoring post-surgical range of motion 238 in users 101 recovering from knee or shoulder arthroplasty. When used by physical therapy clinics delivering physical therapy, the disclosed system 200 can be used for tracking joint condition progress and rehab trajectory, enabling clinicians to see more patients. In those instances, the disclosed system 200 provides objective, quantifiable data to show whether a patient is making progress, staying the same, or declining after surgery and during PT treatment so therapists can adjust treatment plans accordingly.

When used for providing functional movement screen testing, the disclosed system 200 can assist in injury risk identification and mitigation. By tracking joint angles, range of motion, and symmetry with high precision, quantitative analysis helps identify abnormal patterns or compensations that could lead to overuse injuries. Catching these issues early allows for intervention before they become serious.

When used for providing functional movement screen testing, the disclosed system 200 can assist in performance optimization by providing objective data on how efficiently an athlete moves, helping coaches and physical therapists tailor training programs. In those instances, improvements in biomechanics (such as correcting joint misalignments or enhancing mobility) can lead to better performance outcomes (such as increased speed, power, or endurance).

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A method, comprising:
receiving video images of a user from a plurality of video cameras;
using a pre-trained pose detection model to infer landmarks indicative of joints of the user based on the video images of a user;
determining relative locations of the joints of the user in three dimensions based on the landmarks inferred by the pre-trained pose detection model; and
calculating, based on the three-dimensional locations of the joints of the user, qualitative metrics indicative of one or more physiological conditions of the user.

2. The method of claim 1, further comprising:
storing one or more evaluative thresholds indicative of one or more physiological impairments; and
comparing the qualitative metrics of the user to one or more of the evaluative thresholds.

3. The method of claim 2, wherein:
at least one of the qualitative metrics is indicative of a gait of the user; and
at least one of the evaluative thresholds is indicative of an increased probability of a fall.

4. The method of claim 1, further comprising:
storing past qualitative metrics of the user, the past qualitative metrics having been calculated based on past locations of the joints of the user determined using past video images of the user; and
comparing at least some of the qualitative metrics of the user to at least some of the past metrics of the user.

5. The method of claim 4, wherein:
at least one of the qualitative metrics is indicative of a gait speed of the user; and
comparing at least some of the qualitative metrics of the user to at least some of the past metrics of the user comprises determining whether the gait speed of the user has decreased over time.

6. The method of claim 4, further comprising:
displaying, via a graphical user interface, at least some of the qualitative metrics of the user and at least some of the past metrics of the user.

7. The method of claim 1, further comprising:
constructing a digital twin of the user having virtual joints that are separated by distances that are based on the relative locations of the joints of the user.

8. The method of claim 7, further comprising:
storing information indicative of idealized movements for improving one or more physiological conditions of the user;
generating a virtual representation of the digital twin of the user performing one or more of the idealized movements; and
displaying the generated virtual representation overlayed over the captured video images of a user.

9. The method of claim 8, wherein generating the visual representation comprises scaling the one or more idealized movements based on the distances between joints of the user included in the digital twin of the user.

10. The method of claim 9, further comprising:
comparing the relative locations of the joints of the user to relative locations of the virtual joints of the digital twin of the user performing the one or more idealized movements.

11. A system, comprising:
non-transitory computer readable storage media that stores video images of a user received from a plurality of video cameras; and
a hardware computer processor adapted to:
use a pre-trained pose detection model to infer landmarks indicative of joints of the user based on the video images of a user;
determine relative locations of the joints of the user in three dimensions based on the landmarks inferred by the pre-trained pose detection model; and
calculate, based on the three-dimensional locations of the joints of the user, qualitative metrics indicative of one or more physiological conditions of the user.

12. The system of claim 11, wherein:
the computer readable storage media stores one or more evaluative thresholds indicative of one or more physiological impairments; and
the computer processor is further adapted to compare the qualitative metrics of the user to one or more of the evaluative thresholds.

13. The system of claim 12, wherein:
at least one of the qualitative metrics is indicative of a gait of the user; and
at least one of the evaluative thresholds is indicative of an increased probability of a fall.

14. The system of claim 11, wherein:
the computer readable storage media stores past qualitative metrics of the user, the past qualitative metrics having been calculated based on past locations of the joints of the user determined using past video images of the user; and
the computer processor is further adapted to compare at least some of the qualitative metrics of the user to at least some of the past metrics of the user.

15. The system of claim 14, wherein:
at least one of the qualitative metrics is indicative of a gait speed of the user; and
the computer processor is adapted to determine whether the gait speed of the user has decreased over time.

16. The system of claim 14, further comprising:
a graphical user interface adapted to display at least some of the qualitative metrics of the user and at least some of the past metrics of the user.

17. The system of claim 11, wherein the computer processor is further adapted to construct a digital twin of the user having virtual joints that are separated by distances that are based on the relative locations of the joints of the user.

18. The system of claim 17, wherein:
the computer readable storage media stores information indicative of idealized movements for improving one or more physiological conditions of the user; and
the computer processor is further adapted to generate a virtual representation of the digital twin of the user performing one or more of the idealized movements and display the generated virtual representation overlayed over the captured video images of a user.

19. The system of claim 18, wherein the computer processor is adapted to generate the visual representation by scaling the one or more idealized movements based on the distances between joints of the user included in the digital twin of the user.

20. The method of claim 19, wherein the computer processor is adapted to compare the relative locations of the joints of the user to relative locations of the virtual joints of the digital twin of the user performing the one or more idealized movements.
